(19) United States
(12) Patent Application Publication
(10) Pub. No.: US 2021/0350490 A1
(43) Pub. Date: Nov. 11, 2021

(54) CRUISE EMBARKATION SYSTEMS AND METHODS FOR PREVENTION OF DISEASE TRANSMISSION

(71) Applicant: Royal Caribbean Cruises Ltd., Miami, FL (US)

(72) Inventors: Curt Joseph Frey, Miami, FL (US); Derek Andrew Major, Overland Park, KS (US); Daniel David Daley, Miami, FL (US); Daniel John Altin, Miami, FL (US)

(21) Appl. No.: 17/307,878

(22) Filed: May 4, 2021

Related U.S. Application Data
(60) Provisional application No. 63/020,411, filed on May 5, 2020.

(51) Int. Cl. G06Q 50/26; G16H 10/20; G16H 10/60; G16H 15/00; G16H 50/80; G16H 10/40; G06Q 10/10; G06Q 50/14; G16H 40/67

(52) U.S. Cl. CPC G06Q 50/265; G16H 10/20; G16H 10/60; G16H 15/00; G16H 40/67; G16H 10/40; G06Q 10/10; G06Q 50/14; G16H 50/80

(57) ABSTRACT

Embarkation systems and methods for preventing the introduction and/or spread of infectious diseases within a cruise ship or other environment. An embarkation system includes at least one agent computing device located at an embarkation facility and configured to receive a disease-negative input corresponding to a customer's test result or proof of immunity to an infectious disease, retrieve a health status of the customer determined based at least in part on responses to one or more health questions provided to the customer, and to assist in controlling embarkation based at least in part on the disease-negative input and the health status of the customer. The at least one agent computing device may be operated by an agent of the cruise ship and may execute an agent app configured to display instructions and/or customer's boarding approval status to the agent.

### CROSS-REFERENCE TO RELATED APPLICATIONS

This application claims the benefit of U.S. Provisional Application Ser. No. 63/020,411, filed May 5, 2020, titled “CRUISE EMBARKATION SYSTEMS AND METHODS FOR PREVENTION OF DISEASE TRANSMISSION,” which is incorporated by reference herein in its entirety.

### BACKGROUND

### Technological Field

The present application relates to cruise ship embarkation systems and methods for preventing the spread of contagious diseases.

### Description of the Related Art

Cruise ships are often described as floating cities. These ships are designed to provide every convenience and necessity to hundreds and in many cases thousands of passengers during a sailing that can range from 2 days to as many as 4 weeks. Cruise ships need their own autonomous medical facilities, including the infrastructure and data necessary to support those facilities.

### SUMMARY

The system, method, and devices of this disclosure each have several innovative aspects, no single one of which is solely responsible for its desirable attributes disclosed herein. Without limiting the scope of this disclosure, its more prominent features will now be discussed briefly. After considering this discussion, and particularly after reading the section entitled “Detailed Description” one will understand how the features of this disclosure provide advantages over other personalized recommendation solutions.

In a first aspect, an embarkation system for preventing the introduction of an infectious disease to a cruise ship comprises a data store and at least one agent computing device located at an embarkation facility associated with the cruise ship. The data store has stored thereon a plurality of customer profiles associated with individual customers expected to embark on the cruise ship, the data store in communication with a network. Each individual customer is associated with a user computing device of a plurality of user computing devices, each user computing device configured to store customer profile information of a respective customer of the individual customers, each user computing device further configured to provide, to the customer via a graphical user interface, one or more health questions associated with determination of a health status of the customer at a predetermined time prior to expected embarkation of the customer; transmit, to the data store, the health status of the customer determined based at least in part on one or more inputs received from the customer in response to the one or more health questions; and display, on a display of the user computing device, a boarding document associated with the customer. The at least one agent computing device is in communication with the data store and configured to receive a disease-negative input corresponding to at least one of: a test result associated with the customer and the infectious disease, or a proof of immunity to the infectious disease associated with the customer; retrieve, based on the boarding document, the health status of the customer and the indicator of the temperature of the customer from the data store; determine, based at least in part on the health status of the customer, that the customer does not have any predefined symptoms associated with the infectious disease; and display an indication that the customer is allowed to enter a sterile zone of the embarkation facility based at least in part on the disease-negative input.

In some embodiments, each user computing device is further configured to receive, from a computing device associated with the cruise ship, a communication indicating that the customer should not travel to the embarkation facility based on the health status; and provide, to the customer, a notification associated with the communication.

In some embodiments, the embarkation system further comprises a temperature screening device located within a pre-sterile zone of the embarkation facility, the temperature screening device configured to measure a body temperature of the customer and to cause an indicator of the body temperature of the customer to be stored in the data store in association with the health status of the customer. In some embodiments, the at least one agent computing device determines that the customer does not have any predefined symptoms associated with the infectious disease further based at least in part on the indicator of the body temperature of the customer. In some embodiments, at least one agent computing device determines that the customer does not have any predefined symptoms further based at least in part on a temperature threshold defined by a government entity having authority at the embarkation facility or at an intended port of call of the cruise ship.

In some embodiments, the proof of immunity comprises a vaccination record.

In some embodiments, the disease-negative input comprises a negative test result associated with the customer and the infectious disease. In some embodiments, the negative test result is based on a test for the infectious disease administered within a predetermined time period prior to one of an expected time of departure, a time of arrival of the customer at a pre-sterile zone of the embarkation facility, or an arrival window time assigned to the customer. In some embodiments, the negative test result is based on a test for the infectious disease administered within a pre-sterile zone of the embarkation facility. In some embodiments, the negative test result is based on at least one of a molecular diagnostic test and an antigen test associated with the infectious disease.

In some embodiments, each user computing device is further configured to display, to the customer, an embarkation facility arrival window indicating a time period during which the customer will be permitted to enter the embarkation facility.

In some embodiments, the at least one agent computing device is further configured to display an indication that the customer is allowed to enter a pre-sterile zone of the embarkation facility only during the time period indicated by the arrival window.

In some embodiments, the at least one agent computing device is further configured to display, to an agent of an operator of the cruise ship, a boarding approval status corresponding to the customer. In some embodiments, the boarding approval status is an approved status if the disease-negative input is received. In some embodiments, the boarding approval status is a denied status if the disease-negative input is not received and an inconclusive or positive test result is received corresponding to the customer.

In some embodiments, the one or more health questions are generated based at least in part on one or more regulations established by a government entity having authority at the embarkation facility or at an intended port of call of the cruise ship.

In some embodiments, the at least one agent computing device causes the customer to be allowed to enter the sterile zone further based at least in part on one or more other disease-negative inputs corresponding to one or more other customers in a predetermined grouping with the customer. In some embodiments, the predetermined grouping comprises the customers associated with a stateroom of the cruise ship.

In a second aspect, a method for preventing introduction of an infectious disease into a cruise ship environment comprises permitting customers to enter a pre-sterile zone of an embarkation facility, each customer being permitted to enter based on a manifest of customers expected to embark. The method further comprises, within the pre-sterile zone of the embarkation facility, determining a health status of each customer by receiving, from a data store, customer-provided answers to one or more health questions provided to the customer at a predetermined time prior to embarkation, or receiving answers to the one or more health questions from the customer within the pre-sterile zone; and providing a secondary screening for the infectious disease for customers having a health status associated with one or more predefined symptoms of the infectious disease or for customers who have not provided a negative test result associated with the infectious disease. The method further comprises permitting individual customers to pass from the pre-sterile zone to a sterile zone of the embarkation facility based on at least one of: receiving a disease-negative input corresponding to at least one of: a test result associated with the customer and the infectious disease, or a proof of immunity to the infectious disease associated with the customer; determining that an individual customer does not have a health status associated with one or more predefined symptoms of the infectious disease; and determining, based on the secondary screening, that an individual customer does not have the infectious disease or is not a carrier of a pathogen associated with the infectious disease.

In some embodiments, the method further comprises determining, based on the customer-provided answers to the one or more health questions provided to the customer at the predetermined time prior to embarkation, that one or more customers of the customers expected to embark are likely to be denied boarding; and providing an instruction to the one or more customers not to travel to the embarkation facility.

In some embodiments, the method further comprises, within the pre-sterile zone of the embarkation facility, measuring a body temperature of each customer using a temperature screening device. In some embodiments, the method further comprises, within the pre-sterile zone of the embarkation facility, providing a secondary screening for the infectious disease for customers based at least in part on the measured body temperature exceeding a pre-determined temperature threshold. In some embodiments, individual customers are permitted to pass from the pre-sterile zone to the sterile zone of the embarkation facility based on determining that the individual customer does not have a health status associated with the one or more predefined symptoms of the infectious disease only if the individual customer is not determined to have a body temperature exceeding the pre-determined temperature threshold. In some embodiments, the temperature threshold is defined by a government entity having authority at the embarkation facility or at an intended port of call of the cruise ship.

In some embodiments, the one or more health questions are generated based at least in part on one or more regulations established by a government entity having authority at the embarkation facility or at an intended port of call of the cruise ship.

In some embodiments, the proof of immunity comprises a vaccination record.

In some embodiments, the disease-negative input comprises a negative test result associated with the customer and the infectious disease. In some embodiments, the negative test result is based on a test for the infectious disease administered within a predetermined time period prior to one of an expected time of departure, a time of arrival of the customer at a pre-sterile zone of the embarkation facility, or an arrival window time assigned to the customer. In some embodiments, the negative test result is based on a test for the infectious disease administered within a pre-sterile zone of the embarkation facility. In some embodiments, the negative test result is based on at least one of a molecular diagnostic test and an antigen test associated with the infectious disease.

In some embodiments, each customer is admitted to enter the pre-sterile zone based at least in part on the customer arriving at the pre-sterile zone during an arrival window assigned to the customer.

In some embodiments, the secondary screening is provided to all customers.

In some embodiments, the secondary screening comprises an on-site test for the infectious disease.

In some embodiments, each individual customer is allowed to pass from the pre-sterile zone to the sterile zone further based at least in part on one or more other customers in a predetermined grouping with the individual customer being allowed to pass from the pre-sterile zone to the sterile zone. In some embodiments, the predetermined grouping comprises the customers associated with a stateroom of the cruise ship.

### BRIEF DESCRIPTION OF THE DRAWINGS

The disclosed aspects will hereinafter be described in conjunction with the appended drawings and appendices, provided to illustrate and not to limit the disclosed aspects, wherein like designations denote like elements.

FIG. 1 shows a diagram of a networked computing system including a ship-based computing system communicating with a shore-based computing system via a cloud-based or wireless network, according to an exemplary embodiment.

FIGS. 2A and 2B schematically illustrate pre-sterile and sterile zones of an embarkation facility in accordance with the present technology.

FIG. 3 schematically illustrates example embarkation processes in accordance with the present technology.

FIG. 4 schematically illustrates further example embarkation processes in accordance with the present technology.

FIGS. 5A-5F illustrate example graphical user interfaces (GUIs) for implementing embarkation systems and methods in accordance with the present technology.

### DETAILED DESCRIPTION

Generally described, the embarkation systems and methods described herein provide for the division of an embarkation facility into at least a sterile zone and a pre-sterile zone such that all customers must pass through the pre-sterile zone before entering the sterile zone where they can embark. Within the pre-sterile zone, and prior to admission into the sterile zone, each customer can be evaluated to determine whether the customer has a particular infectious disease. Customers are prevented from passing into the sterile zone if it cannot be determined that they do not have the disease. The present disclosure provides testing protocols administered within the pre-sterile zone, as well as additional network-enabled verification steps prior to arrival at an embarkation facility, which provide for efficient handling of customers within the pre-sterile zone. Moreover, some embodiments of the systems and methods of the present disclosure allow customers to expedite their passage through the pre-sterile zone based on their level of participation prior to arriving at the embarkation facility.

Although embodiments of the embarkation systems and methods described herein are described in the context of embarkation of customers of a cruise operator, it will be understood that the present technology is not limited to this class of customer, this type of service provider, or the particular cruise context. As will be described in detail below, features of the present technology can be employed in many other contexts, such as but not limited to entertainment, hotel, and other hospitality services and systems; festivals or other large public events or gatherings; other transportation systems such as air, rail, or road transport providers (e.g., airlines, passenger railroads, intercity bus lines, and the like); healthcare services and systems; and health information technology (HIT) services and systems.

The health and wellness-related embarkation systems and methods of the present technology may in some embodiments be used to provide pre-boarding benefits to guests. For example, as described herein, embarkation processes may include pre-boarding aspects such as a health questionnaire to be completed by guests within a predetermined time window (e.g., 24 hours) prior to boarding (e.g., delivered through a mobile application or browser-based web application associated with the cruise operator). In such embodiments, if a guest responds that they are suffering from one or more predefined symptoms associated with a targeted illness, 24 hours before their cruise, the system can provide suggestions to shore-side customer care agents who can help guests avoid being turned away at the terminal. In some cases, guests reporting certain symptoms or combinations of symptoms may be instructed not to travel to the terminal and board the cruise. Thus, the systems and methods described herein can allow guests whose health conditions may prevent them from traveling to be refunded and/or otherwise compensated for canceled travel and avoid a difficult or time-consuming experience at the terminal if they ultimately will not be embarking. Moreover, risk of exposure is reduced for guests who do embark, as they will not be exposed to the ill guests who do not travel to the embarkation facility. For guests who do travel, embarkation processes with health screening features can be expedited by the systems and methods of the present technology. Additionally, embodiments of the health and wellness-related embarkation systems and methods of the present technology use valuable resources and capacity at the terminal facility much more efficiently, because guests who are sick or likely to be sick are identified and rebooked before the embarkation process begins. Conservation of limited, and in some cases costly, embarkation-related resources for guests who are, or are likely to be, disease-free speeds the embarkation process, contributing to guest satisfaction and shorter turnaround times and other costs savings for the cruise operator.

Embodiments of the present technology use various databases to store health information corresponding to customers. In the context of embarkation processes for a cruise experience, a customer can be, but is not limited to, a potential customer that has not yet booked a cruise, a customer who has booked a cruise on a sailing that has not yet begun, and a customer that is a passenger on a particular sailing of a ship in real-time. Moreover, it will be understood that in the context of the embarkation processes described herein, customers may include passengers or guests of the cruise operator as well as crew members or other agents of the cruise operator.

### Exemplary Term Descriptions

To facilitate an understanding of the systems and methods discussed herein, a number of terms are described below. The terms described below, as well as other terms used herein, should be construed to include the provided descriptions, the ordinary and customary meaning of the terms, and/or any other implied meaning for the respective terms. Thus, the descriptions below do not limit the meaning of these terms, but only provide exemplary definitions.

Data Store: Includes any computer readable storage medium and/or device (or collection of data storage mediums and/or devices) that stores data. Examples of data stores include, but are not limited to, optical disks (for example, CD-ROM, DVD-ROM, and so forth), magnetic disks (for example, hard disks, floppy disks, and so forth), memory circuits (for example, solid state drives, random-access memory (“RAM”), and so forth), and/or the like. Another example of a data store is a hosted storage environment that includes a collection of physical data storage devices that may be remotely accessible and may be rapidly provisioned as needed (commonly referred to as “cloud” storage).

Database: Includes any data structure (and/or combinations of multiple data structures) for storing and/or organizing data, including, but not limited to, relational databases (for example, graph databases, Oracle databases, MySQL databases, and so forth), non-relational databases (for example, NoSQL databases, and so forth), in-memory databases, spreadsheets, as comma separated values (“CSV”) files, Extensible Markup Language (“XML”) files, text (“TXT”) files, flat files, spreadsheet files, and/or any other widely used or proprietary format for data storage. Databases are typically stored in one or more data stores. Accordingly, each database referred to herein (for example, in the description herein and/or the figures of the present application) is to be understood as being stored in one or more data stores.

### Example Computing Systems of Embarkation Systems and Methods of the Present Technology

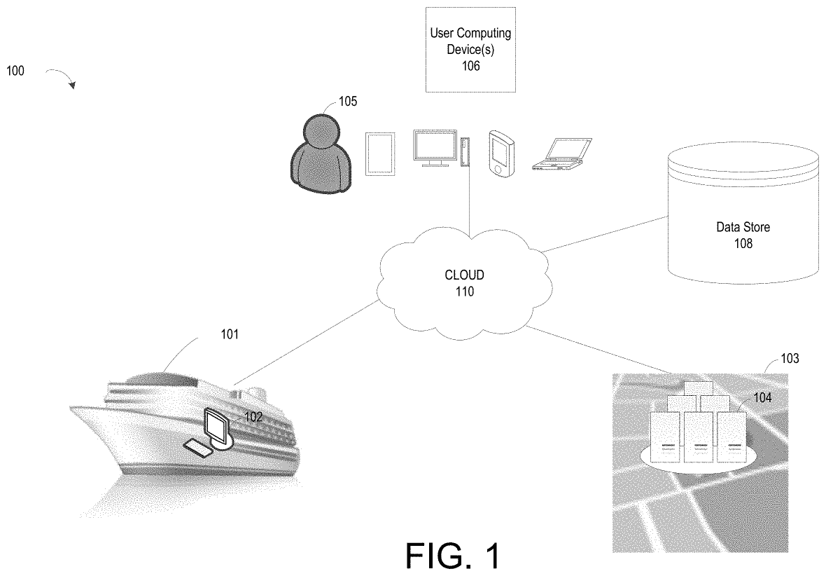

FIG. 1 illustrates a diagram of an example networked computing system 100 of the systems and methods described herein. The system 100 includes a ship-based computing system 102 communicating with a shore-based computing system 104 via a cloud-based or wireless network 110, according to this exemplary embodiment. In some embodiments, the ship-based computing system 102 is located on a ship 101 (or other mass transport, such as a train, a shuttle, a bus, a plane, and so forth) configured to transport a plurality of individuals or customers (“customers”) during a trip or voyage (“sailing”). The shore-based computing system 104 is located in a shore-based facility 103 and is associated with a service provider, such as a company or entity that operates the ship 101. Alternatively, or additionally, the shore-based computing system 104 may include a cloud-based database or data store 108 with which the ship-based computer system 102 communicates at least periodically while in port or at sea. Alternatively or in addition, the shore-based computing system 104 may include a cloud-based computing system with which the ship-based computer system 102 communicates at least periodically.

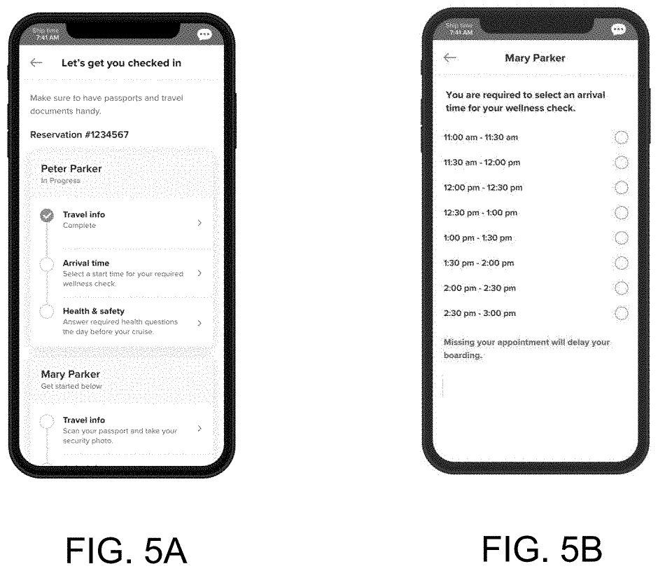

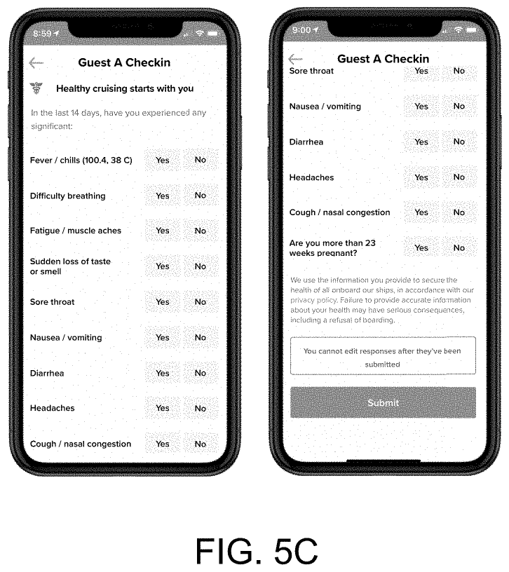

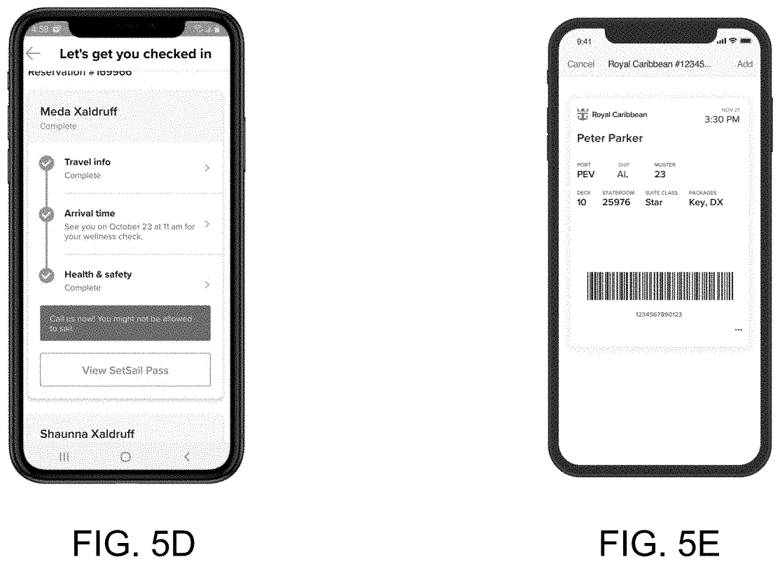

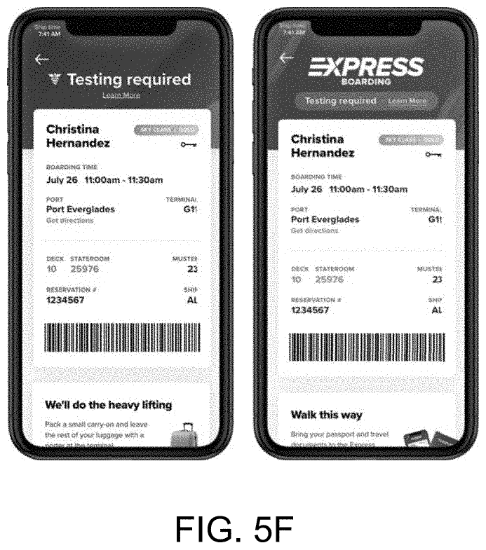

The system 100 also includes a plurality of user computing devices 106 that are operated by customers of the service provider. The user computing devices 106 may include any one of a smartphone, a tablet, a laptop, a desktop, a personal digital assistant (PDA), a vehicle communication system, a smartwatch, or any other electronic device. The user computing devices 106 may have installed thereon one or more applications such as an embarkation application or other application associated with the cruise provider, which allows a user 105 (for example, the customer, as described above) to perform a variety of operations associated with embarkation. For example, the one or more applications may provide an interface through which a customer may enter travel information (e.g., personal, security, and/or health information), choose a preselected arrival time or arrival time window for embarkation, provide health and safety information such as answers to questions associated with a health status of the customer, provide proof of immunity to one or more known diseases, request and/or view a boarding pass, receive push notifications associated with the customer's cruise and/or embarkation, communicate with the cruise operator, etc. Further details of cruise operator-associated applications executing on user computing devices 106 are described in greater detail with reference to FIGS. 5A-5F.

Implementations of the present technology advantageously store various types of data in databases associated with the shore-based computing system 104 and the ship-based computing system 102 of each ship in the cruise operator's fleet. For example, the shore-based computing system 104 may store data associated with all ships owned or operated by the cruise operator; each ship's previously-offered sailings, sailings that are currently underway in real-time, and future, scheduled sailings.

The shore-based computing system 104 and/or the ship-based computing system 102 can also store data relating to each customer that is embarked on or is scheduled to embark on a particular sailing offered by the cruise operator. The shore-based computing system 104 can also store data relating to each customer that previously sailed on, is currently embarked on, or is scheduled to sail on a particular sailing operated by the cruise operator. In some examples, the shore-based computing system 104 stores historical information such as health information for customers that have purchased cruise services over the last year, 2 years, 3 years, 4 years, 5 years, or any other suitable length of time. Such information may be stored in accordance with any applicable data privacy and/or health information privacy requirements, and may be maintained in accordance with requirements or mandates of relevant health-related organizations such as the Centers for Disease Control and Prevention (CDC) or other agencies. Moreover, health information for one or more individual customers may be removed from the ship-based and/or shore-based computing systems 102, 104 if requested by the corresponding customers.

In some embodiments, the ship-based computing system 102 stores data associated with customers that are embarked on a particular sailing of the ship 101. The ship-based computing system 102 may also store data associated with the ship 101, the sailing, and health data associated with customers on the sailing (for example, results of health questions, documented immunity status, and/or health screening data). Some or all of this information can be transferred to the ship-based computing system 102 from the data store 108, or another data source. In some examples, the information is transferred to the ship-based computing system 102 close in time to the beginning of a particular sailing, such as 1 week, 6 days, 5 days, 4 days, 3 days, 2 days, 1 day, 12 hours, 8 hours, 6 hours, 4 hours, 3 hours, 2 hours, or 1 hour before the ship embarks on the sailing, or any other suitable time.

### Example Embarkation Processes of the Present Technology

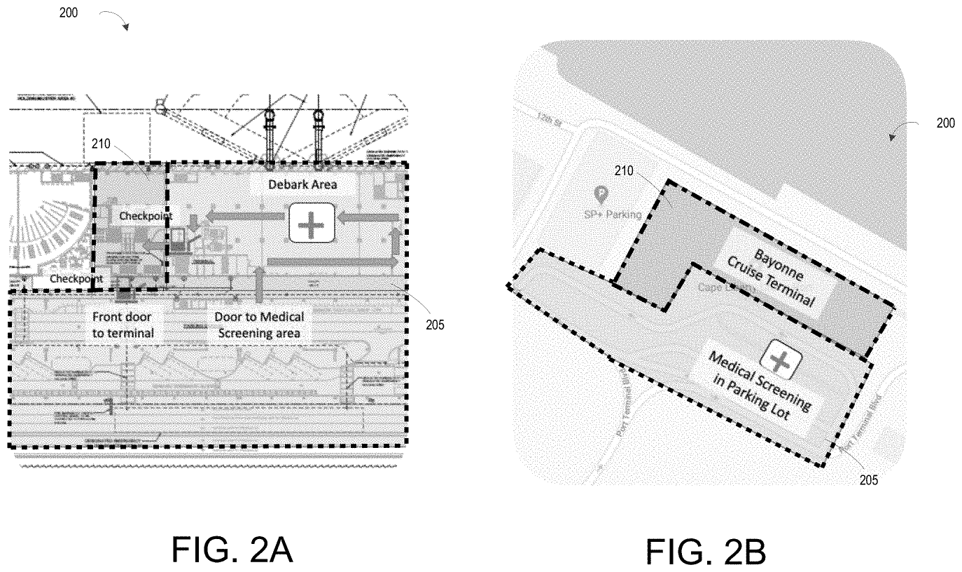

FIGS. 2A and 2B illustrate example pre-sterile and sterile zones of an embarkation facility 200 such as a cruise terminal or the like. As shown in FIGS. 2A and 2B, a sterile zone 210 may be separated from a pre-sterile zone 205, such as by a physical barrier (e.g., a wall, a fence, separate rooms, visual indicators, or the like), and/or by personnel of the cruise operator.

The sterile zone 210 (also referred to herein as a “green zone”) may be the final portion of the embarkation facility entered by customers. For example, customers may board a cruise ship directly from the sterile zone 210. The pre-sterile zone 205 (also referred to herein as a “yellow zone”) may be adjacent to the sterile zone 210 and may be a more public-facing portion of the embarkation facility 200. For example, the pre-sterile zone 205 may include a parking lot, a portion of a cruise terminal building having one or more exterior doors, or any other suitable location that can be controlled (physically and/or operationally) relative to the sterile zone 210. Customer access to the sterile zone 210 from the pre-sterile zone 205 may generally be limited to those customers determined to be free of one or more identified infectious diseases, as will be described in greater detail. In some embodiments, the pre-sterile zone 205 may further be access-restricted, and may be divided from an exterior or “red zone” by a checkpoint such that only customers appearing on a ship manifest for an intended sailing may enter, and the general public may be prevented from entering the pre-sterile zone 205.

In some embodiments, the sterile zone 210 may include services and facilities such as waiting areas, baggage handling facilities, and the like. The pre-sterile zone 205 may include additional services and facilities such as waiting areas, temperature screening systems and devices, medical screening facilities, third party medical testing personnel, etc.

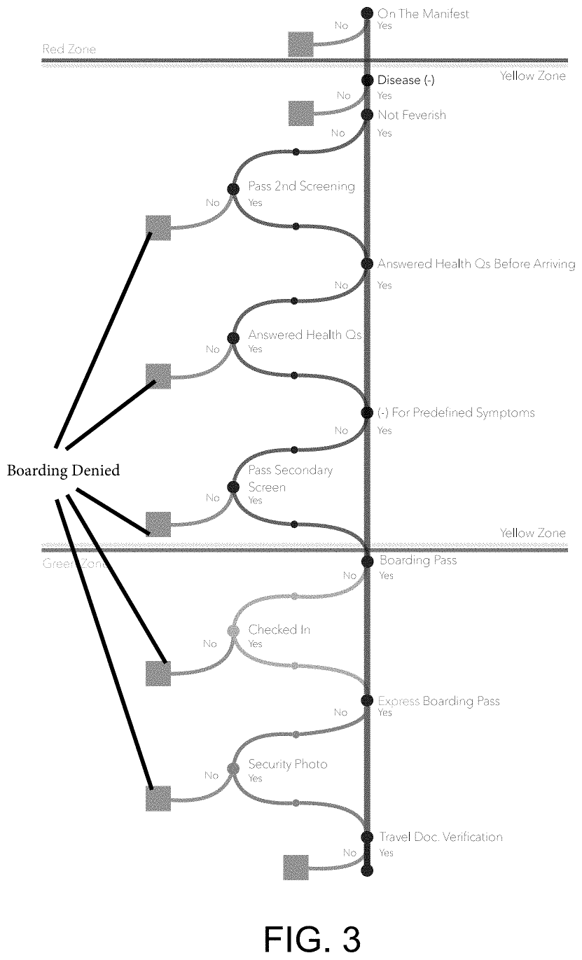

Referring now to FIG. 3, example embarkation systems and processes will now be described. The schematic flow diagram of FIG. 3 provides a series of checkpoints by which each customer arriving at an embarkation facility may be screened and ultimately allowed to embark if the customer passes the required health screenings and is determined to not be infected with a particular infectious disease (or has met threshold requirements deemed by applicable health authorities to be safe for boarding). Although the embarkation processes will be described with reference to a single example customer and a single disease, it will be understood that the processes described herein may generally be applied to all customers intending to embark, and may in some cases be applied for a plurality of known diseases simultaneously.

Embodiments of the embarkation process are described with reference to determining that a customer is free of one or more predetermined infectious diseases. Absolute or near certainty that the customer is not infected with a predetermined infectious disease is desirable and advantageously achievable using embodiments of the present disclosure. It will be understood, however, that the present disclosure can also be implemented in which a risk that a customer is infected with a particular infectious disease is assessed relative to a spectrum of risks or “go/no-go” criteria. For example, embodiments of the present disclosure can include a spectrum including low, medium, and high risk of infection criteria, and determine a risk an individual customer presents relative to the spectrum using information gleaned from the customer's health check in the pre-sterile zone. In one non-limiting embodiment, customers assessed to have a zero or low risk of being infected with an infectious disease are admitted to the green zone, customers assessed to have a medium risk of being infected with the infectious disease are subjected to secondary screening or rebooked on a future sailing, and customers assessed to have a high risk of being infected with the infectious disease are rebooked on a future sailing. The number of risk levels can be more or less than those described in this example. Further, the outcomes associated with each risk level may be adjusted based on the characteristics of the predetermined infectious disease, such as transmission and mortality rate.

The embarkation process may begin within the red zone at the step labeled “On the Manifest,” where an arriving customer may first be evaluated to determine if the customer is on the manifest for a voyage. For example, the customer may present an identification document such as a passport or driver license, may present a printed or mobile boarding pass, or may simply provide the customer's name and any other requested identifying information. In some embodiments, a mobile boarding pass may be generated by a cruise operator application executing on the customer's mobile device as described with reference to FIG. 1. Admission to the pre-sterile zone may be further conditioned on one or more criteria in addition to appearing on a manifest. For example, in some embodiments, customers may be assigned arrival windows indicating a time period during which they will be admitted to the embarkation facility. Enforcement of arrival windows may advantageously ensure that customer arrivals occur at a predictable rate, avoiding the buildup of large groups within the pre-sterile zone or within the exterior red zone. Arrival windows may be assigned automatically to customers, who may be notified of their assigned arrival windows via an application executing on a user device. In some embodiments, customers may be provided an option to select an available arrival window in the application prior to traveling to the embarkation facility. An example arrival window selection screen is illustrated in FIG. 5B. If it is determined that the customer is on the manifest and/or has arrived at the embarkation facility within a pre-assigned arrival window, the customer may be allowed to proceed from the red zone into the yellow zone, or pre-sterile zone.

Within the pre-sterile zone, one or more health checks may be performed in order to ensure that the customer is free of one or more predetermined infectious diseases, prior to being admitted to the green zone, or sterile zone. As will be described in greater detail, if a customer does not pass one or more of the health checks in the pre-sterile zone, the customer may be directed to a secondary screening such that the customer may still be allowed to embark if it can be determined that the customer does not have the predetermined infectious disease despite failing one or more of the health checks.

As an initial step, the customer is administered a test for the infectious disease, as indicated by the “Disease (−)” checkpoint within the yellow zone in FIG. 3. In some embodiments, every customer entering the pre-sterile zone is tested for the infectious disease using an industry-standard test known to detect the particular infectious disease. Any suitable test can be implemented in embodiments of the present disclosure. Example tests include any suitable point-of-care (molecular or antigen) test, including nucleic acid amplification tests (NAAT) such as but not limited to polymerase chain reaction (PCR) tests, antigen tests such as but not limited to lateral flow assays, and the like. Any immunoassay test or diagnostic test with a suitable response time may be employed. The testing may be performed by the cruise operator or by a third party infectious disease testing provider. The results of the on-site test are preferably rapid (e.g., result provided within 60 minutes or less, 40 minutes or less, 30 minutes or less, 20 minutes or less, 10 minutes or less, 5 minutes or less, 1 minute or less, etc.). In some embodiments, the testing may be performed in conjunction with a network-enabled test reader device which may wirelessly transmit the test results to the cruise provider (e.g., to an application executing on a cruise operator agent computing device) and may optionally transmit the results to the customer's computing device. If the result of the test is negative, the customer may be considered to have a low risk of having or carrying the infectious disease, and may be required to undergo further screening steps as a precaution to detect erroneous or false negative test results. If the result of the test is positive, the customer may be considered to have a high risk of having or carrying the infectious disease, and may be required to undergo further screening steps as a precaution to detect erroneous or false positive test results.

In one non-limiting example, some customers entering the pre-sterile zone are not tested for the infectious disease. In some embodiments, a verified proof of immunity to the infectious disease (e.g., a vaccination record showing the customer received a vaccination for the infectious disease or a record of a positive antibody test corresponding to the infectious disease) may allow the customer to proceed to the sterile zone, either with no further screening or with a streamlined screening. Customers providing verified proof of immunity prior to arrival at the pre-sterile zone may receive verification from the cruise operator that on-site testing will not be administered, for example in the form of a mobile “no test required” boarding pass. Example customer app screens illustrating different arrival testing requirements are illustrated in FIG. 5F. In some implementations, a customer providing proof of immunity (prior to arrival at the pre-sterile zone or upon arrival at the pre-sterile zone) may simply be screened for an elevated body temperature and allowed to pass to the green zone if an elevated body temperature is not detected.

In one example implementation, any person seeking to enter the sterile zone is required to provide a “negative for disease” credential. The negative-for-disease credential can include, for example, proof of immunity (in the form of a vaccination record or a positive antibody test result) or a negative result of an on-site test performed in the pre-sterile zone. In this example, admittance into the sterile zone is denied in the absence of the negative-for-disease credential. The negative-for-disease credential can be applicable to all individuals (including cruise operator employees, third party vendors, inspectors, etc.) at each request to enter the sterile zone.

Further health screenings can be performed after the customer is confirmed to possess a “negative-for-disease” credential. As an example health screening step, at the “Not Feverish” checkpoint indicated in FIG. 3, all customers may be screened using a temperature screening device to detect any customers having a fever. The temperature screening device may be any suitable temperature measurement device, and in some embodiments may include one or more thermal imaging devices. Customers determined not to have an elevated body temperature (e.g., a body temperature below a predetermined fever threshold) may pass to a next screening step or to the green zone. Customers determined to have an elevated body temperature or fever (e.g., a body temperature above the predetermined fever threshold) may be sent to a secondary screening in which one or more other tests may be performed. The predetermined fever threshold may be determined, for example, based on fever thresholds suggested or required in accordance with one or more regulations enacted by a governmental entity having authority at the location of the embarkation facility and/or at the location of one or more intended ports of call for the cruise ship.

At the secondary screening, a customer having a fever may be tested using one or more other testing methods, such as disease-specific tests configured to determine whether the customer is infected with the disease or a pathogen associated with the disease, and/or to determine whether the customer has antibodies associated with the disease. If it is determined in the secondary screening that the customer does not have the disease, the customer may pass to the next screening step or to the green zone. If it is determined in the secondary screening that the customer does have the disease, the customer may be denied embarkation in order to avoid infecting other customers.

In another example health screening step, customers may be screened based on their answers to one or more questions associated with the infectious disease. For example, the answers to the questions may indicate whether the customer has any symptoms that are known to be associated with the infectious disease. If the customer has previously answered the questions (e.g., by completing an app-based questionnaire as shown, for example, in FIG. 5C and as indicated at the “Answered health Qs Before Arriving” checkpoint in FIG. 3), the customer's answer may be evaluated to determine if the customer has any predefined symptoms associated with the infectious disease. If the customer has not previously provided answers, the customer may be required to answer the questions within the pre-sterile zone before proceeding. If the customer's answers to the questions indicate that the customer does not have symptoms associated with the infectious disease, the customer may be permitted to pass to a subsequent screening step or to the green zone. If the customer's answers to the questions indicate that the customer does have one or more symptoms associated with the infectious disease, the customer may be sent to a secondary screening, which in some cases may be the same secondary screening used for passengers determined to have a fever (e.g., as shown in the at-risk status screen illustrated in FIG. 5D). The questions provided to the customers may be determined and/or selected, for example, based on questions suggested or required in accordance with one or more regulations enacted by a governmental entity having authority at the location of the embarkation facility and/or at the location of one or more intended ports of call for the cruise ship. In one example implementation, if local authorities at the embarkation facility and at one or more different ports of call require different sets of pre-embarkation questions to be asked of embarking customers, the set of all required questions of the different local authorities may be included in the set of questions asked to each customer before arrival via user computing devices and/or in the pre-sterile zone.

If the customer does not have any of the predefined symptoms (as indicated by the “(−) For Predefined Symptoms” checkpoint in FIG. 3) and/or passes a secondary screening, the customer may then be permitted to pass to the green zone. Within the green zone, one or more final embarkation processes may occur. For example, a customer who has not yet checked in and received a boarding pass may do so, a security photo may be taken, travel documents such as a government-issued identification may be verified, etc. The customer may then be permitted aboard the ship with relative certainty that the customer is not bringing the identified infectious disease aboard.

In various implementations, the question-based screening may be performed before or after temperature screening of guests. In some embodiments, the question-based screening may be performed at least in part outside of the pre-sterile zone, such as prior to arrival at the embarkation facility. In one example, the relevant questions may be presented to the customer at a predetermined time, such as approximately 24 hours, prior to the planned embarkation in the form of a questionnaire to be completed by the customer through an application executing on the customer's computing device. The customer may answer the questions through a graphical user interface(e.g., as shown in FIG. 5C). The application may then send the answers, or a health status determined based on the answers, to a data store associated with the cruise operator, where the user's answers or health status may be stored in a customer profile.

In further embodiments, the customer may receive notifications associated with the customer's health status prior to arriving at the embarkation facility. In one example, if the customer completes the health questionnaire 24 hours before embarkation and provides answers that indicate the customer likely has the infectious disease, the customer may be instructed not to travel to the embarkation facility (e.g., by a push notification, email, phone call, text message, etc. to the mobile device), and/or to contact the cruise operator to determine whether the customer should travel to the embarkation facility, and may be offered a refund (e.g., as shown in FIG. 5D). Although it is not desirable to prevent a customer from embarking on a planned voyage, it may be significantly more convenient for a customer to be informed and offered a refund the day before a planned embarkation, rather than wasting the time associated with traveling to and waiting at an embarkation facility before being denied embarkation within the pre-sterile zone.

A customer's path through the pre-sterile and sterile zones may be different based on the level of participation of the customer prior to the day of embarkation. There may be several possible paths, ranging from a “non-engager” path, in which a customer is booked on a sailing but has not checked in, received a boarding pass, or provided answers to health questions prior to arriving at the embarkation facility, to an “express boarding pass” path, in which a customer is able to travel through the embarkation facility in minimal time.

A “non-engaging” customer arriving at the embarkation facility may first be required to present identification to confirm that the customer is on the manifest, and then may be required to take a test for the infectious disease, undergo a temperature screening, provide a series of answers to health questions, and undergo any necessary secondary screening prior entering the sterile zone. Within the sterile zone, the non-engaging customer may further be required to check in to receive a boarding pass, have a security photo taken, and have travel documents verified prior to embarking.

In contrast, a customer who has engaged with the embarkation process prior to the day of embarkation may have a more expedited path through the embarkation facility. For example, if the customer checks in using the application on the customer's computing device, answers the health questions within the application, and is determined not have any predefined symptoms of the infectious disease based on the answers to the questions, the customer may be issued a mobile boarding pass (e.g., as shown in FIG. 5E) which allows the customer to enter the pre-sterile zone and pass to the sterile zone by only being tested for the infectious disease and passing through a temperature screening process. Having already checked in, the customer may travel quickly through the sterile zone without requiring an additional check-in process, and may embark after having a security photo taken. In some embodiments, where proof of immunity is available such as by a vaccination record or proof of a positive antibody test, a customer may receive an express boarding pass prior to the day of embarking. With the express boarding pass, the customer may be permitted to pass through the pre-sterile zone without undergoing a temperature screening or any secondary screening processes.

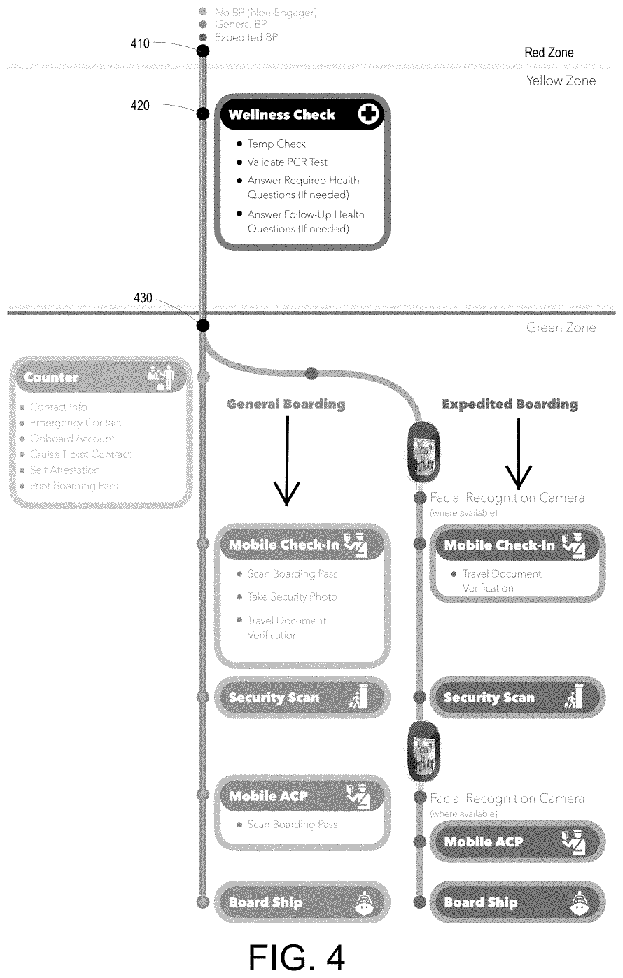

Referring now to FIG. 4, further example embarkation processes will be described. In the example of FIG. 4, all customers proceed to a wellness check in the yellow or pre-sterile zone before proceeding according to a participation-based one of a plurality of boarding processes within the green or sterile zone.

At step 410 customers arrive at the embarkation facility at an exterior or red zone, outside the pre-sterile zone or yellow zone. If customers have pre-assigned arrival windows, an agent may be present at the entry to the pre-sterile zone to control entry to the pre-sterile zone. The agent may operate an agent computing device running an application (e.g., an “agent app”) configured to display information to the agent regarding customers assigned to the current arrival window. For example, in some embodiments, the agent app may display a list of the customers assigned to the current arrival window. In some embodiments, the agent app may additionally or alternatively allow the agent to input a name, scan an identity document or boarding pass, etc., and responsively provide an indication of whether the identified customer is on the manifest and assigned to the current arrival window. If the agent app indicates that the customer is not assigned to the current arrival window, the agent app may provide further information such as an indication that the customer is not on the manifest, an indication that the customer has been denied boarding (e.g., based on a previous positive test for the infectious disease or based on one or more previous health question answers), or an indication of the correct arrival window assigned to the customer if the customer has simply arrived at the wrong time. If the customer has arrived after the assigned arrival window, the agent app may include functionality to reassign the customer to a subsequent arrival window with availability remaining. If the agent app indicates that the customer is assigned to the current arrival window, the agent app may direct the agent to allow the customer to enter. In some embodiments, the agent app may identify groupings of customers such as all customers associated with an individual stateroom, and may require all customers of the individual grouping to be present before the customers are admitted to the pre-sterile zone, such that the customers of each grouping are admitted as a group for the wellness check.

At step 420, each customer or grouping of customers proceeds to a wellness check. In some embodiments, the wellness check includes a temperature check for each individual customer. A body temperature may be determined for each individual customer using any suitable temperature measurement device. In some embodiments, a contactless temperature measurement device, such as an infrared thermometer, thermal imaging device, or the like may be used to determine the body temperature of each customer. The body temperature of each customer may then be compared to a predetermined fever threshold to determine if the customer is feverish. The fever threshold may be determined, for example, based on any of various accepted sources of medical guidance, such as guidance from the World Health Organization (WHO), the Centers for Disease Control (CDC), or other suitable source. In some embodiments, the threshold may be determined based on a threshold established as suggested or required by a government entity having authority at the location of the embarkation facility and/or at the location of one or more intended ports of call for the cruise ship.

In some embodiments, the wellness check includes a test for the infectious disease administered to all customers. The test may employ any suitable point-of-care testing method (molecular or antigen) as described elsewhere herein. In some embodiments, the biological sample provided by each customer for the test may be associated with a test identifier associated with the customer in a database such as the data store 108 (FIG. 1), such that the customer or an agent may be notified electronically (e.g., via a user computing device or an agent app on an agent computing device) of each customer's test result (or a health-related notice indicative of the test result described in more details below, for example “denied to board following test result” or “cleared to board following test result”).

In some embodiments, the wellness check includes asking individual customers some or all of the required pre-embarkation health questions as described elsewhere herein. For example, if the customer failed to answer some or all of the questions prior to arriving at the embarkation facility, an agent within the pre-sterile zone may ask the customer the required questions and input the answers using the agent app on an agent computing device. If any of the customer's previously provided answers indicated that the customer may possibly be infected with the infectious disease, or if the customer was found to have a body temperature above the predetermined fever threshold, the customer may further be asked to answer one or more follow-up health questions to further assess whether the customer has the infectious disease.

After providing any information and/or biological samples required for the wellness check, customers may wait within the pre-sterile zone until the results of the infectious disease test results are available. In some embodiments, individual customers and/or groups of customers corresponding to predefined groupings (e.g., individual stateroom groups) may be notified in sequence of a boarding approval status for the customer or for the group of customers. For example, if the customer and/or if each customer in the customer's grouping satisfies the criteria for admission to the sterile zone (e.g., the customer and/or all customers in the customer's grouping have negative test results from the infectious disease test administered in step 420 and/or are not feverish and did not provide any questionnaire answers indicating possible infection), the customer or grouping of customers may be notified by an agent and directed to proceed to step 430, where they pass from the pre-sterile zone into the sterile zone. Upon entry to the sterile zone 430, each customer can proceed to embark by one of a plurality of boarding processes, such as a general boarding process, an expedited boarding process, or a non-engager boarding process, based on an amount of engagement or participation of the customer prior to arrival at the embarkation facility.

In some cases, a customer's test results from the test administered at step 420 may not be negative. For example, a customer may receive a positive test result indicating the customer has the infectious disease, or may receive an inconclusive test result indicating that the test could not determine whether the customer has the infectious disease. In some embodiments, in case of either a positive or inconclusive test result, the agent app of an agent computing device operated by an agent in the pre-sterile zone may indicate a “denied” boarding approval status. The agent may communicate the denied status to the customer and direct the customer (and optionally the grouping of customers including the customer having an inconclusive or positive test result) to leave the pre-sterile zone and return to the red zone or exterior of the embarkation facility. The use of a “denied” boarding approval status, rather than informing the agent of the actual positive or inconclusive test result, may desirably provide better protection of the customer's confidential medical information as determined by the test processing entity. In some embodiments, a customer having an inconclusive test result may alternatively be allowed to retake the infectious disease test prior to being denied boarding.

The processes described herein with reference to FIGS. 3 and 4 may each be performed at least partially in conjunction with an application executing on one or more networked computing devices operated by agents of the cruise operator. For example, when a customer receives a positive or negative test result, answers health questions, or receives a result of any secondary screening, the results may be input into the application by an agent. Referring again to FIG. 1, the information received during the embarkation process, as well as any additional information provided by customers through the application executing on their computing devices, may be sent to the shore-based computing system 104 and/or the data store 108. The shore-based computing system 104 and/or the data store 108 may store such information in association with customer profiles associated with the customers. The shore-based computing system 104 and/or the data store 108 may additionally store information such as sailing manifests which may be utilized in verifying that customers arriving at the embarkation facility are on the manifest for a particular sailing.

In some embodiments, the health data received for the customers may be sent to the ship-based computing system 102 at some point prior to sailing. For example, the health data may be transferred from the shore-based computing system 104 or from the data store 108 to the ship-based computing system 102, or may be sent to the ship-based computing system 102 directly from customers' user computing devices 106 and/or the computing devices operated by agents of the cruise line as information is received at those devices prior to embarkation. Preferably, the health data gleaned from the customers is stored within the ship-based computing system 102 such that it is accessible to onboard health professionals and facilities during the sailing.

Referring again to FIGS. 2A and 2B, embodiments of the health and wellness-related embarkation systems and methods of the present technology can prevent or reduce cross-contamination between the pre-sterile zone 205 and the sterile zone 210. In one non-limiting example, flow between the pre-sterile zone 205 and the sterile zone 210 is strictly one way, with customers who are admitted to the sterile zone 210 not permitted to re-enter the pre-sterile zone 205. In another non-limiting example, cruise operator personnel are assigned to process customers in either the pre-sterile zone 205 or the sterile zone 210, but not both, for the duration of the entire embarkation process of a single sailing. Personnel who are assigned to the sterile zone 210 can be required to complete, prior to guest arrival, the above-described testing and health screenings in the pre-sterile zone 205 before gaining entry to the sterile zone 210.

FIGS. 5A-5F illustrate example graphical user interfaces (GUIs) that may be presented to customers and/or agents using a customer app or agent app executing on a user computing device or an agent computing device, respectively, in the implementation of the embarkation systems and methods of the present technology. The particular graphical and textual content of the example GUIs of FIGS. 5A-5F is provided by way of non-limiting example only, and it will be understood that various implementations of the present technology may include GUIs differing from those in FIGS. 5A-5F in visual appearance and textual content without departing from the scope of the present technology. Any of the GUI screens illustrated in FIGS. 5A-5F may be displayed, for example, in a customer app executing on a user computing device 106 as illustrated in FIG. 1. In some embodiments, some or all of the screens illustrated in FIGS. 5A-5F may also be displayable within an agent app executing on an agent computing device operated by an agent of the cruise ship, for example, during a check-in or wellness check procedure implemented at an embarkation facility as described herein. It will be understood that the agent app may include some or all of the same GUI features illustrated and described with regard to the customer app, with additional and/or different textual content provided, such as indications or instructions to the agent using the device, relating to an individual customer or group of customers.

FIG. 5A illustrates an example customer check-in screen of a GUI in accordance with the present technology. The customer check-in screen may be made available for display beginning at a predetermined time prior to embarkation. For example, the customer check-in screen may be made available within a customer app beginning 72 hours prior to a planned embarkation or sailing time, 48 hours prior to a planned embarkation or sailing time, 36 hours prior to a planned embarkation or sailing time, 24 hours prior to a planned embarkation or sailing time, or any other suitable time in advance of the customer's expected arrival at the embarkation facility. In some embodiments, the customer check-in screen is made available at least the day before the planned embarkation so that customers may plan their embarkation day schedules around an assigned arrival window. The customer check-in screen may provide access to an arrival window selection screen, a health and questionnaire screen, and/or a boarding document display screen. In some embodiments, the customer check-in screen may also provide options for upload of disease-negative indication-related documents, such as documentary evidence of a negative test result or vaccination against the infectious disease.

FIG. 5B illustrates an example arrival window selection screen of a GUI in accordance with the present technology. As shown in FIG. 5B, a customer using the customer app may be provided with a plurality of selectable arrival windows, which may be, for example, 2-hour time windows, 90-minute time windows, 60-minute time windows, 30-minute time windows, 15-minute time windows, or other suitable duration. In some embodiments, the selectable arrival window options may be presented based on availability. For example, the customer app on a user computing device 106 (FIG. 1) may access arrival window availability data (e.g., stored in data store 108) to determine which arrival windows are available to the customer at the time of check-in and which arrival windows no longer have availability. Arrival windows that are fully booked may be greyed out and non-selectable, or may not be presented to the customer in the arrival window selection screen.

FIG. 5C depicts an example health questionnaire screen of a GUI in accordance with the present technology. The health questionnaire screen provides one or more health questions that are associated with the determination of the health status of the customer. In some embodiments, the health questionnaire screen may provide a list of symptoms, each associated with selectable “yes” and “no” options for the customer to indicate whether the customer has the symptom or if the customer has had the symptom within a predetermined time period before answering the questionnaire. In some embodiments, the health questionnaire screen is only made available to the customer at a predetermined time prior to embarkation, such as the day before embarkation, so as to ensure that the answers to the health questions are reliable on the day of embarkation. In some embodiments, the health questionnaire screen may be accessible through an agent app on an agent computing device operated by an agent of the cruise ship operator, such as to guide a customer through the health questions at the embarkation facility. For example, if a customer has not completed the health questions prior to traveling to the embarkation facility, the agent may ask the customer the questions verbally and input the answers through a GUI such as the health questionnaire screen of FIG. 5C.

FIG. 5D illustrates an example screen indicating a possible at-risk health status associated with a customer. In some embodiments, if a customer answers “yes” to one or more, or another predetermined number, of the health questions, there may be a risk that the customer is infected with the infectious disease. In some cases a subset of the health questions may be sufficient individually to result in an at-risk status (e.g., presence of a fever or sudden loss of taste or smell), while other health questions may not be sufficient individually to result in an at-risk status (e.g., headaches or a sore throat). As shown in FIG. 5D, if a customer's answers to the health questions indicate a possible at-risk status, the customer may be directed to contact the operator of the cruise ship to determine whether or not the customer will be allowed to embark.

FIG. 5E illustrates an example boarding document display screen of a GUI in accordance with the present technology. The displayed boarding document may include identifying information such as the customer's name, embarkation facility identifier, ship identifier, stateroom identifier, and any other appropriate information. A computer-readable identifier, such as a barcode, QR code, or the like, may also be provided. Accordingly, upon arrival at the entrance to a pre-sterile zone of an embarkation facility, a customer may present the boarding document display screen to an agent. The agent may use an agent computing device executing an agent app to scan the computer-readable identifier. The agent app may then retrieve customer profile information corresponding to the boarding document to determine if the customer is on an appropriate manifest and has arrived during the customer's assigned arrival window. If the customer is on the manifest and has arrived during the assigned arrival window, the agent app can display an indication to the agent that the customer is allowed to enter the pre-sterile zone.

In some embodiments, a plurality of types of electronic boarding documents may be available based on a vaccination or testing status of the customer. FIG. 5F illustrates two example boarding document screens that may be displayed in a customer app on a user computing device. A “testing required” boarding document may be displayed if the customer has not uploaded or otherwise provided proof of a recent negative test result or a vaccination against the infectious disease. Alternatively, if the customer has uploaded proof of a recent negative test result or a vaccination record indicating that the customer is vaccinated against the infectious disease, the electronic boarding document may be an “express boarding” document that may allow the customer to bypass some or all of the wellness check steps described herein.

### Additional Embodiments

It will be understood that not necessarily all objects or advantages may be achieved in accordance with any particular embodiment described herein. Thus, for example, those skilled in the art will recognize that certain embodiments may be configured to operate in a manner that achieves or optimizes one advantage or group of advantages as taught herein without necessarily achieving other objects or advantages as may be taught or suggested herein.

Embarkation systems and methods described herein provide for a safe embarkation process that prevents the introduction of pathogens and/or diseases into a cruise ship environment. Advantageously, the embarkation systems and methods described herein are deployable to any number of ships in the cruise operator's fleet. This allows the operator to leverage information that the operator has developed across its entire line of ships for all customers, in a way that does not require data-intensive computing resources to be deployed on each individual ship. Embarkation systems and methods of the present technology are also uniquely suited to facilitate health tracking in the ship-to-shore ecosystem in which cruise ships operate, because they can push health data to a ship at times when the ship is connected to the shore framework and then use the ship's framework to continue health monitoring autonomously at sea, even when the ship's data services are not connected to the shore framework (via the cloud or any other mechanism).

With reference again to FIG. 1, in implementations of the present technology, the ship 101 (and the ship-based computing system 102) have different levels of connectivity with the shore-based facility 103 (and the shore-based computing system 104) at different times in the customer's cruise lifecycle. For example, when the ship 101 is not underway (for example, before and/or after a sailing), the ship 101 may be docked in a home port and have a wired or wireless connection with the shore-based computing system 104. When the ship 101 is underway (for example, at sea enroute to ports of call on the sailing), the ship 101 (and the ship-based computing system 102) may have, at best, satellite communications with the shore-based facility 103 (and the shore-based computing system 104). When the ship 101 is docked in a port other than its homeport during the sailing, the ship 101 (and the ship-based computing system 102) may have satellite or land-based communications with the shore-based facility 103 (and the shore-based computing system 104).

In some embodiments, the communications that occur when the ship 101 is underway or when the ship 101 is docked in a visiting port during the sailing are significantly reduced as compared to those communications that occur when the ship 101 is docked in its homeport. For example, the communications that occur when the ship 101 is docked in the visiting port or when the ship 101 is underway may have one or more of reduced speed, bandwidth, and/or reliability for at least a portion of time when the ship 101 is underway or in the visiting port. In some embodiments, communications between the ship-based computing system 102 and the shore-based computing system 104 are discontinued altogether while the ship 101 is underway or in the visiting port. For example, the communications are discontinued or reduced when the ship 101 is traveling in geographic regions or areas where even satellite and/or other long-range communications are unavailable or limited (for example, when the ship 101 is at open sea out of telecommunications range with shore-based facilities and satellite communications are unavailable or not economically feasible). In some embodiments, the ship 101 has access to home-based cellular communications, Wi-Fi communications, and so forth when docked in its homeport. In some embodiments, the ship 101 has access to cellular communications, Wi-Fi communications, and so forth when docked in a port of call, such that the ship 101 may have access to information stored in the data store 108 via the cloud 110. In some embodiments, the ship 101 has access to satellite, long-range communications, and so forth when underway between ports.

In some embodiments, the least expensive, most reliable communications between the ship 101 and the shore-based facility 103 occur over the home-based cellular communications, Wi-Fi communications, and so forth available in the ship's home port. At such times, communications between the ship 101 and the shore-based facility 103 may also offer the highest data speeds and bandwidth. The most expensive, least reliable communications between the ship 101 and the shore-based facility 103 may occur over the satellite or other long-range communication links that are available while the ship 101 is underway between ports. For example, the satellite or other long-range communications may be expensive and/or provide limited speeds or bandwidths for data communications. Thus, data communications between the shore-based facility 103 and the ship 101 using the home-based communications may be the most expedient or practical method of communication of the available options. Remote cellular communications, Wi-Fi communications, and other forms of communication available in a port of call may be equivalent to or substantially similar to home-based communications, or they may be significantly worse or completely unavailable. Satellite or other long-range communications may be the least expedient or practical method of communicating, if they are even available. In some cases, depending on geographic location and costs of access, communication between the shore-based facility 103 and the ship 101 is simply not possible using any of the above-described communication links.

In some embodiments, the ship-based computing system 102 and/or the shore-based computing system 104 is configured to determine whether the ship is in the homeport, sailing between ports, or docked in a port of call. Accordingly, the ship-based computing system 102 or the shore-based computing system 104 may determine a state of connectivity between the ship- and shore-based computing systems 102 and 104, respectively.

Many other variations than those described herein will be apparent from this disclosure. For example, depending on the embodiment, certain acts, events, or functions of any of the algorithms described herein can be performed in a different sequence, can be added, merged, or left out altogether (for example, not all described acts or events are necessary for the practice of the algorithms). Moreover, in certain embodiments, acts or events can be performed concurrently, for example, through multi-threaded processing, interrupt processing, or multiple processors or processor cores or on other parallel architectures, rather than sequentially. In addition, different tasks or processes can be performed by different machines and/or computing systems that can function together.

The elements of a method, process, or algorithm described in connection with the embodiments disclosed herein can be embodied directly in hardware, in a software module stored in one or more memory devices and executed by one or more processors, or in a combination of the two. A software module can reside in RAM memory, flash memory, ROM memory, EPROM memory, EEPROM memory, registers, hard disk, a removable disk, a CD-ROM, or any other form of non-transitory computer-readable storage medium, media, or physical computer storage known in the art. An example storage medium can be coupled to the processor such that the processor can read information from, and write information to, the storage medium. In the alternative, the storage medium can be integral to the processor. The storage medium can be volatile or nonvolatile. The processor and the storage medium can reside in an ASIC. The ASIC can reside in a user terminal. In the alternative, the processor and the storage medium can reside as discrete components in a user terminal.

All of the methods and processes described herein may be embodied in, and partially or fully automated via, software code modules executed by one or more general purpose computers. For example, the methods described herein may be performed by the computing system and/or any other suitable computing device. The methods may be executed on the computing devices in response to execution of software instructions or other executable code read from a tangible computer readable medium. A tangible computer readable medium is a data storage device that can store data that is readable by a computer system. Examples of computer readable mediums include read-only memory, random-access memory, other volatile or non-volatile memory devices, CD-ROMs, magnetic tape, flash drives, and optical data storage devices.

What is claimed is:

1. An embarkation system for preventing the introduction of an infectious disease to a cruise ship, the embarkation system comprising:
a data store having stored thereon a plurality of customer profiles associated with individual customers expected to embark on the cruise ship, the data store in communication with a network;
each individual customer associated with a user computing device of a plurality of user computing devices, each user computing device configured to store customer profile information of a respective customer of the individual customers, each user computing device further configured to:
provide, to the customer via a graphical user interface, one or more health questions associated with determination of a health status of the customer at a predetermined time prior to expected embarkation of the customer;
transmit, to the data store, the health status of the customer determined based at least in part on one or more inputs received from the customer in response to the one or more health questions; and
display, on a display of the user computing device, a boarding document associated with the customer;
at least one agent computing device located at an embarkation facility associated with the cruise ship, the at least one agent computing device in communication with the data store and configured to:
receive a disease-negative input corresponding to at least one of: a test result associated with the customer and the infectious disease, or a proof of immunity to the infectious disease associated with the customer;
retrieve, based on the boarding document, the health status of the customer from the data store;
determine, based at least in part on the health status of the customer, that the customer does not have any predefined symptoms associated with the infectious disease; and
display an indication that the customer is be allowed to enter a sterile zone of the embarkation facility based at least in part on the disease-negative input.

2. The embarkation system of claim 1, wherein each user computing device is further configured to:
receive, from a computing device associated with the cruise ship, a communication indicating that the customer should not travel to the embarkation facility based on the health status; and
provide, to the customer, a notification associated with the communication.

3. The embarkation system of claim 1, further comprising a temperature screening device located within a pre-sterile zone of the embarkation facility, the temperature screening device configured to measure a body temperature of the customer and to cause an indicator of the body temperature of the customer to be stored in the data store in association with the health status of the customer.

4. The embarkation system of claim 3, wherein the at least one agent computing device determines that the customer does not have any predefined symptoms associated with the infectious disease further based at least in part on the indicator of the body temperature of the customer.

5. The embarkation system of claim 4, wherein at least one agent computing device determines that the customer does not have any predefined symptoms further based at least in part on a temperature threshold defined by a government entity having authority at the embarkation facility or at an intended port of call of the cruise ship.

6. The embarkation system of claim 1, wherein the proof of immunity comprises a vaccination record.

7. The embarkation system of claim 1, wherein the disease-negative input comprises a negative test result associated with the customer and the infectious disease.

8. The embarkation system of claim 7, wherein the negative test result is based on a test for the infectious disease administered within a predetermined time period prior to one of an expected time of departure, a time of arrival of the customer at a pre-sterile zone of the embarkation facility, or an arrival window time assigned to the customer.

9. The embarkation system of claim 7, wherein the negative test result is based on a test for the infectious disease administered within a pre-sterile zone of the embarkation facility.

10. The embarkation system of claim 7, wherein the negative test result is based on at least one of a molecular diagnostic test and an antigen test associated with the infectious disease.

11. The embarkation system of claim 1, wherein each user computing device is further configured to display, to the customer, an embarkation facility arrival window indicating a time period during which the customer will be permitted to enter the embarkation facility.

12. The embarkation system of claim 1, wherein the at least one agent computing device is further configured to display an indication that the customer is allowed to enter a pre-sterile zone of the embarkation facility only during the time period indicated by the arrival window.

13. The embarkation system of claim 1, wherein the at least one agent computing device is further configured to display, to an agent of an operator of the cruise ship, a boarding approval status corresponding to the customer.

14. The embarkation system of claim 13, wherein the boarding approval status is an approved status if the disease-negative input is received.

15. The embarkation system of claim 13, wherein the boarding approval status is a denied status if the disease-negative input is not received and an inconclusive or positive test result is received corresponding to the customer.

16. The embarkation system of claim 1, wherein the one or more health questions are generated based at least in part on one or more regulations established by a government entity having authority at the embarkation facility or at an intended port of call of the cruise ship.

17. The embarkation system of claim 1, wherein the at least one agent computing device causes the customer to be allowed to enter the sterile zone further based at least in part on one or more other disease-negative inputs corresponding to one or more other customers in a predetermined grouping with the customer.

18. The embarkation system of claim 17, wherein the predetermined grouping comprises the customers associated with a stateroom of the cruise ship.

19. A method for preventing introduction of an infectious disease into a cruise ship environment, the method comprising:
permitting customers to enter a pre-sterile zone of an embarkation facility, each customer being permitted to enter based on a manifest of customers expected to embark;
within the pre-sterile zone of the embarkation facility:
determining a health status of each customer by:
receiving, from a data store, customer-provided answers to one or more health questions provided to the customer at a predetermined time prior to embarkation, or
receiving answers to the one or more health questions from the customer within the pre-sterile zone; and
providing a secondary screening for the infectious disease for customers having a health status associated with one or more predefined symptoms of the infectious disease or for customers who have not provided a negative test result associated with the infectious disease; and
permitting individual customers to pass from the pre-sterile zone to a sterile zone of the embarkation facility based on at least one of:
receiving a disease-negative input corresponding to at least one of: a test result associated with the customer and the infectious disease, or a proof of immunity to the infectious disease associated with the customer;
determining that an individual customer does not have a health status associated with one or more predefined symptoms of the infectious disease; and
determining, based on the secondary screening, that an individual customer does not have the infectious disease or is not a carrier of a pathogen associated with the infectious disease.

20. The method of claim 19, further comprising:
determining, based on the customer-provided answers to the one or more health questions provided to the customer at the predetermined time prior to embarkation, that one or more customers of the customers expected to embark are likely to be denied boarding; and
providing an instruction to the one or more customers not to travel to the embarkation facility.

21. The method of claim 19, further comprising, within the pre-sterile zone of the embarkation facility, measuring a body temperature of each customer using a temperature screening device.

22. The method of claim 21, further comprising, within the pre-sterile zone of the embarkation facility, providing a secondary screening for the infectious disease for customers based at least in part on the measured body temperature exceeding a pre-determined temperature threshold.

23. The method of claim 22, wherein individual customers are permitted to pass from the pre-sterile zone to the sterile zone of the embarkation facility based on determining that the individual customer does not have a health status associated with the one or more predefined symptoms of the infectious disease only if the individual customer is not determined to have a body temperature exceeding the pre-determined temperature threshold.

24. The method of claim 22, wherein the pre-determined temperature threshold is defined by a government entity having authority at the embarkation facility or at an intended port of call of the cruise ship.

25. The method of claim 19, wherein the one or more health questions are generated based at least in part on one or more regulations established by a government entity having authority at the embarkation facility or at an intended port of call of the cruise ship.

26. The method of claim 19, wherein the proof of immunity comprises a vaccination record.

27. The method of claim 19, wherein the disease-negative input comprises a negative test result associated with the customer and the infectious disease.

28. The method of claim 27, wherein the negative test result is based on a test for the infectious disease administered within a predetermined time period prior to one of an expected time of departure, a time of arrival of the customer at a pre-sterile zone of the embarkation facility, or an arrival window time assigned to the customer.

29. The method of claim 27, wherein the negative test result is based on a test for the infectious disease administered within a pre-sterile zone of the embarkation facility.

30. The method of claim 27, wherein the negative test result is based on at least one of a molecular diagnostic test and an antigen test associated with the infectious disease.

31. The method of claim 19, wherein each customer is admitted to enter the pre-sterile zone based at least in part on the customer arriving at the pre-sterile zone during an arrival window assigned to the customer.

32. The method of claim 19, wherein the secondary screening is provided to all customers.

33. The method of claim 19, wherein the secondary screening comprises an on-site test for the infectious disease.

34. The method of claim 19, wherein each individual customer is allowed to pass from the pre-sterile zone to the sterile zone further based at least in part on one or more other customers in a predetermined grouping with the individual customer being allowed to pass from the pre-sterile zone to the sterile zone.

35. The method of claim 34, wherein the predetermined grouping comprises the customers associated with a stateroom of the cruise ship.
